(19) United States
(12) Patent Application Publication
(10) Pub. No.: US 2006/0094695 A1
(43) Pub. Date: May 4, 2006

(54) Hypoestoxides, derivatives and agonists thereof for use in the prophylaxis and treatment of obesity

(75) Inventors: Emmanuel A. Ojo-Amaize, Glendora, CA (US); Emeka J. Nchekwube, Morgan Hill, CA (US); Donna N. Nchekwube, Morgan Hill, CA (US)

Correspondence Address: PILLSBURY WINTHROP SHAW PITTMAN LLP, P.O BOX 10500, McLean, VA, 22102, US

(73) Assignee: Paraquest, Inc., Bloomington, CA (US)

(21) Appl. No.: 10/979,613

(22) Filed: Nov. 2, 2004

(51) Int. Cl. A61K 31/665; A61K 31/60; A61K 31/5377; A61K 31/454; A61K 31/336

(52) U.S. Cl. 514/100; 514/475; 514/232.5; 514/165; 514/323

(57) ABSTRACT

Methods of treating a host suffering from obesity or an overweight condition are provided. Methods of preventing obesity or an overweight condition are also provided. The methods include delivery of a hypoestoxide, hypoestoxide derivative or hypoestoxide agonist, or hypoestes dried leaf powder to a host suffering from obesity or an overweight condition or a host at risk for developing obesity or becoming overweight. The methods further include the administration of a chemotherapeutic agent, such as a non-steroidal anti-inflammatory drug, in combination with delivery of a hypoestoxide, hypoestoxide derivative or hypoestoxide agonist, or hypoestes dried leaf powder.

### FIELD OF THE INVENTION

This invention relates to the use of diterpene compounds for the treatment and prophylaxis of obesity. In particular, disclosed herein are hypoestes rosea dried leaf powder, hypoestoxides, derivatives and agonists thereof for the treatment and prophylaxis of obesity and being overweight.

### CROSS REFERENCE TO RELATED APPLICATIONS

Expressly incorporated by reference as if fully set forth herein are: U.S. Provisional Pat. App. No. 60/491,683, U.S. Pat. Nos. 5,801,193, 5,994,328, 6,001,871, and 6,242,484, and co-pending applications, U.S. Ser. Nos. 09/006,946; 09/007,308; 09/298,653; and PCT WO 98/46222.

### BACKGROUND OF THE INVENTION

Obesity is an excess of body fat frequently resulting in a significant impairment of health. Obesity results when the size or number of fat cells in a person's body increases. A non-obese person has between 30 and 35 billion fat cells. When a person gains weight, these fat cells increase in size, and in cases of progressive weight gain, in number as well. One pound of body fat represents about 3500 calories. When a person loses weight, fat cells decrease in size, but the number of fat cells generally stays constant.

Body Mass Index (BMI), expressed as weight/height² (BMI=kg/m²), is commonly used to classify individuals 20 years of age and older as overweight (BMI=25.0-29.9) and obese (BMI=30 or over) (Allison, D., et al., Annual deaths attributable to obesity in the U.S., Journal of the American Medical Association, 1999, Vol. 282, pp. 1530-1538). Obesity is considered an epidemic in the U.S., with a prevalence of approximately 20% (Mokdad, AH, et al; The continuing epidemics of obesity and diabetes in the U.S. JAMA 2001, 286: pp. 1195-1200). The annual healthcare costs associated with obesity are estimated to exceed $100 billion (Allison, D., et al; Annual deaths attributable to obesity in the U.S. JAMA 1999, 282: pp. 1530-1538).

Approximately 300,000 people die of obesity-related diseases each year (Allison, D., et al; Annual deaths attributable to obesity in the U.S. JAMA 1999, 282: pp. 1530-1538). Patients with BMIs that exceed 30 are at risk for significant co-morbidities such as type 2 diabetes, heart and kidney disease, hypertension, respiratory diseases, dyslipidemia, sleep apnea, orthopedic problems, gall bladder disease, certain cancers and osteoarthritis (Must, A., JAMA 1999, 282: pp. 1523-1529). Both morbidity and mortality are increased in obese individuals (Lisser, L., et al; Variability of body weight and health outcomes in the Framingham population. New England Journal of Medicine 1995, 333: pp 677-685). Mortality from cardiovascular disease rises with increasing BMI (Stevens, J., et al; The effect of age on the association between body-mass index and mortality. New England Journal of Medicine 1998, 338(1): pp 1-7). The risk of coronary artery disease doubles at BMI greater than 27 and is nearly quadrupled if the index is greater than 32. These risks are worsened by smoking (Jung, RT., Obesity as a disease. British Medical Bulletin 1997, 53(2): pp 307-321).

Anti-obesity drugs can be classified into two broad groups: (i) those acting on the central nervous system (“CNS”) to influence appetite and satiety; and (ii) those acting on the gastrointestinal tract to reduce absorption.

CNS anti-obesity drugs have included fenfluramine and dexfenfluramine, which were effective weight-reduction drugs that have been withdrawn from the worldwide market due to complications including primary pulmonary hypertension and hypertropic cardiac valvular lesions. Ephedrine and caffeine have primarily anorectic properties, although some thermogenic effects have been demonstrated (Astrup et al., The effect and safety of an ephedrine/caffeine compound compared to ephedrine, caffeine and placebo in obese subjects on an energy-restricted diet. Int. J. Obes., 1992, 16(4), pp. 269-77). Phentermine and diethylproprion are amphetamine derivatives that are effective at suppressing appetite and reducing weight, but because of their stimulant action on the CNS, only short term use, for less than 3 months, is recommended. Sibutramine is a serotonin that enhances post-digestive satiety and increases resting metabolic rates in animals, but there have been reports of increased blood pressure and heart rates in some patients.

Gastrointestinal tract anti-obesity drugs have included metformin, which may be useful in managing obesity in those with type 2 diabetes and in those with impaired glucose tolerance. However, if used in subjects with cardiac decomposition, renal disease or hepatic disease, it may result in lactic acidosis. Orlistat is a pancreatic lipase inhibitor which produces a dose-dependent reduction in dietary fat absorption. However, malabsorption of fat-soluble vitamins has been observed.

Thus, many of the conventional drug treatments for obesity have unwanted side-effects. Accordingly, there is a need for additional drug approaches to the treatment and prevention of obesity.

### SUMMARY OF THE INVENTION

The use of hypoestoxides and hypoestoxide derivatives offer a novel drug approach to the treatment and prevention of obesity. Hypoestoxides operate by novel mechanisms such as angiogenesis inhibition (Ojo-Amaize, E., et al., Hypoestoxide, a natural nonmutagenic diterpenoid with antiangiogenic and antitumor activity: Possible mechanisms of action. Cancer Research, 2002, Vol. 62, pp. 4007-4014). Because adipose tissue growth is angiogenesis dependent, adipose tissue mass can be regulated through the vasculature Rupnick, M. A. et al., Adipose tissue mass can be regulated through the vasculature. PNAS 2002, vol. 99. no. 16, pp. 10730-10735). Hypoestoxide is also a prototype of a novel class of IKK inhibitors (Ojo-Amaize, E., et al., Hypoestoxide, a novel anti-inflammatory natural diterpene, inhibits the activity of IkB kinase. Cellular Immunology, 2001, Vol. 209, No. 2, pp. 149-157). Targeted disruption of IKK has been shown to induce reversal of obesity (Yuan, M. et al., Reversal of obesity- and diet-induced insulin resistance with salicylates or targeted disruption of IkkB. Science, 2001, Vol. 293, pp. 1673-1677).

The present invention provides methods of treating a host, such as a human, suffering from being overweight or obese, with hypoestes rosea dried leaf powder, or hypoestoxides, derivatives and agonists thereof, such that the obesity or overweight condition is ameliorated. Thus, the methods include delivery of a therapeutically or prophylactically effective amount of a compound of formula I: 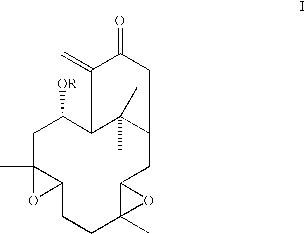
wherein R is:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or
i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms,
wherein the effective amount is an amount sufficient to ameliorate or prevent at least one aspect of obesity or of an overweight condition, and wherein the compound may be used alone or in combination with other chemotherapeutic agents.

### DETAILED DESCRIPTION OF THE INVENTION

As used herein, the term “host” or “subject” is taken to mean human, as well as other animals. The term “ameliorate” means to improve, lessen the severity of or mitigate.

Methods of treating a host suffering from obesity or an overweight condition are provided. Methods of preventing obesity or overweight conditions are also provided. In one method, an effective amount of hypoestes dried leaf powder, a hypoestoxide, hypoestoxide derivative, or an agonist thereof, or a compound of formula I is delivered to a host suffering from obesity or an overweight condition in an amount sufficient to ameliorate at least one aspect of the obesity or overweight condition. In another method, an effective amount of hypoestes dried leaf powder, a hypoestoxide, hypoestoxide derivative, or an agonist thereof, or a compound of formula I is delivered to a host at risk of developing obesity or an overweight condition in an amount sufficient to prevent at least one aspect of the obesity or overweight condition. In another method, an effective amount of hypoestes dried leaf powder, a hypoestoxide, hypoestoxide derivative, or an agonist thereof, or a compound of formula I is delivered to a host suffering from obesity or an overweight condition in an amount sufficient to ameliorate at least one aspect of the obesity or overweight condition, and in conjunction with a standard chemotherapy regimen including, but not in any way limited to, a non-steroidal anti-inflammatory drug such as aspirin or sodium salicylate. In another method, an effective amount of hypoestes dried leaf powder, a hypoestoxide, hypoestoxide derivative, or an agonist thereof, or a compound of formula I is delivered to a host at risk of developing obesity or an overweight condition in an amount sufficient to prevent at least one aspect of the obesity or overweight condition, and in conjunction with a standard chemotherapy regimen including, but not in any way limited to, a non-steroidal anti-inflammatory drug such as aspirin or sodium salicylate. 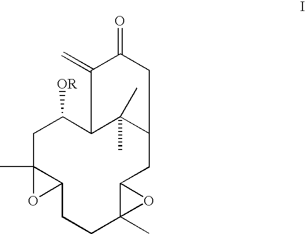
wherein R is:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, or
i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms.

Preferred compounds of the invention are compounds of formula I, wherein R=H and R=acetyl (hypoestoxide).

Hypoestes rosea is the natural source for compounds of the formula I. Hypoestes rosea dried leaf powder contains several components, including hypoestoxides. A typical composition of hypoestes rosea dried leaf powder would be as follows:

| Compound | Prevalence in hypoestes rosea (%) |
|---|---|
| Hypoestoxide | 1 |
| Roseanolone | 1 |
| Chlorophyll and Carotenoids | 75 |
| Minor terpenes and hydrocarbons | 15 |
| Flavonoids and related compounds | 8 |

The magnitude of a prophylactic or therapeutic dose of compounds of formula I in the treatment or prevention of obesity and overweight conditions will vary with the progression of the disease, the chemotherapeutic agent(s) or other therapy used, and the route of administration. The dose, and perhaps the dose frequency, will also vary according to the age, body weight, and response of the individual patient. In general, the total daily dose range for compounds of formula I, or of hypoestes dried leaf powder, for the conditions described herein, is from about 0.5 mg to about 5000 mg, in single or divided doses. Preferably, a daily dose range should be about 1 mg to about 4000 mg, in single or divided doses. In managing the patient, the therapy should be initiated at a lower dose and increased depending on the patient's global response. It is further recommended that infants, children, patients over 65 years, and those with impaired renal or hepatic function initially receive lower doses, and that they be titrated based on global response and blood level. It may be necessary to use dosages outside these ranges in some cases. Further, it is noted that the clinician or treating physician will know how and when to interrupt, adjust or terminate therapy in conjunction with individual patient response. The term “an effective amount” is encompassed by the above-described dosage amounts and dose frequency schedule.

Any suitable route of administration may be employed for providing the patient with an effective dosage of compounds of formula I or of hypoestes dried leaf powder. For example, oral, rectal, parenteral (subcutaneous, intravenous, intramuscular), intrathecal, transdermal, and like forms of administration may be employed. Dosage forms include tablets, troches, dispersions, suspensions, solutions, capsules, patches, and the like. The compound may be administered prior to, concurrently with, or after administration of other chemotherapy, or continuously, i.e., in daily doses, during all or part of, a chemotherapy regimen, such as a TNP-470, aspirin, salicylate, angiostatin, endostatin, Bay-129566 or thalidomide regimen. The compound, in some cases, may be combined with the same carrier or vehicle used to deliver the other chemotherapeutic agent.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound or the crude dried leaf powder of the parent plant may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically or prophylactically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrated agent such as corn starch, potato starch, alginic acid and the like; a lubricant such as magnesium stearate; and a sweetening agent such as sucrose, fructose, lactose or aspartame; or a flavoring agent such as peppermint, oil of wintergreen, or cherry flavoring. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a non-toxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, non-toxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, such as, for example, aluminum monostearate and gelatin.

Sterile injectable solutions are prepared by incorporating the active compound in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of formula I or of hypoestes dried leaf powder can be determined by comparison to their in vivo activity in animal models. Methods for the extrapolation of effective dosages in mice, and other animals, to humans are known in the art (see, for example, U.S. Pat. No. 4,938,949).

The ability of hypoestoxides and hypoestes rosea dried leaf powder to prevent obesity was studied. Hypoestoxide and hypoestes rosea dried leaf powder was administered to C57BL/6J-Lep^{ob} mice. The mice are homozygous for the obese spontaneous mutation and are first recognizable as such at approximately 4 weeks of age. At 4 weeks, the mice weigh 20-25 grams, and may reach three to four times the weight of wild-type mice. Three 4-week-old female mice were placed in each of four treatment conditions, for a total of twelve mice in the study: (i) control; (ii) hypoestoxide was administered orally at a dose of 10 mg/kg; (iii) hypoestoxide was administered orally at a dose of 100 mg/kg; or (iv) hypoestes dried leaf powder was administered orally at a dose of 100 mg/kg. Treatments were administered once a day and the study continued for 21 days. The results of the study are shown in Table 1.

TABLE 1
Prevention of Obesity in Mice

| | Days | | | | | | |
|---|---|---|---|---|---|---|---|
| | −1 | 7 | 14 | 21 | 28 | 35 | 100 |
| Control | 23.9 ± 0.8 | 25.0 ± 3.4 | 30.1 ± 5.2 | 32.8 ± 6.1 | 35.6 ± 8.2 | 37.1 ± 7.4 | 46.9 ± 3.6 |
| Hypoestoxide 100 mg/kg | 24.3 ± 1.1 | 20.9 ± 1.2 | 23.8 ± 0.6 | 27.0 ± 0.8 | 26.4 ± 2.2 | 29.0 ± 2.9 | 40.0 ± 1.5 |
| Hypoestoxide 10 mg/kg | 21.0 ± 1.4 | 19.6 ± 2.0 | 25.4 ± 1.6 | 27.5 ± 1.5 | 28.8 ± 2.3 | 30.3 ± 3.2 | 41.6 ± 2.0 |
| Hypoestes dried leaf | 21.9 ± 1.4 | 18.5 ± 0.7 | 19.8 ± 2.5 | 24.5 ± 3.5 | 24.7 ± 0.6 | 26.0 ± 1.2 | 37.8 ± 1.1 |
| powder 100 mg/kg | | | | | | | |

Treatment of obesity with hypoestoxide and hypoestes rosea dried leaf powder was also studied. Hypoestoxide and hypoestes rosea dried leaf powder was administered to C57BL/6J-Lep^{ob} mice. The mice were treated at approximately 4 months of age at a weight of approximately 50 grams. One mouse was placed in each of three treatment conditions: (i) control; (ii) hypoestoxide was administered orally at a dose of 10 mg/kg; or (iii) hypoestes dried leaf powder was administered orally at a dose of 100 mg/kg. Treatments were administered once a day, with hypoestoxide treatment lasting 50 days and hypoestes dried leaf powder treatment lasting 21 days. The results of the study are shown in Table 2.

TABLE 2
Treatment of Obesity in Mice

| | Days | | | | | | |
|---|---|---|---|---|---|---|---|
| | −1 | 7 | 14 | 21 | 28 | 35 | 160 |
| Control | 50.0 | 50.0 | 50.3 | 51.1 | 52.0 | 55.0 | 76.5 |
| Hypoestoxide | 53.0 | 53.0 | 51.5 | 51.0 | 50.4 | 50.0 | 69.8 |
| 10 mg/kg | | | | | | | |
| Hypoestes | 49.0 | 49.0 | 49.4 | 50.0 | 50.3 | 50.5 | 55.5 |
| dried leaf | | | | | | | |
| powder | | | | | | | |
| 100 mg/kg | | | | | | | |

All publications, patents, and patent documents are incorporated by reference herein, as though individually incorporated by reference.

While the description above refers to particular embodiments of the present invention, it should be readily apparent to people of ordinary skill in the art that a number of modifications may be made without departing from the spirit thereof. The accompanying claims are intended to cover such modifications as would fall within the true spirit and scope of the invention. The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method of prevention and/or treatment of obesity or an overweight condition comprising:
delivering an effective amount of a compound having the formula:

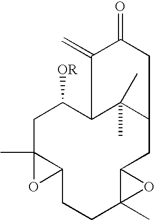

2. The method of claim 1, wherein R is selected from the group consisting of:
a) H or acetyl,
b) P(O)(OH)₂,
c) P(O)(OH)(OM), wherein M is selected from the group consisting of an alkali metal salt and an alkaline earth metal salt,
d) P(O)OM₂ wherein M is each independently selected from the group consisting of alkali metal salts and alkaline earth metal salts,
e) Alkyl of 1 to 12 carbon atoms having 0 to 6 double bonds, said alkyl selected from the group consisting of substituted, unsubstituted, straight chain and branched alkyls,
f) (CH₂)n morpholine, wherein n=1-4,
g) morpholinomethylphenyl, ortho-aminophenyl or ortho-hydroxyphenyl,
h) (CH₂)n COOR₂ wherein n=1-4, R₂ is each selected from the group consisting of H, an alkali metal salt, an alkaline earth metal salt, NH₄+ and N+(R₃)₄ wherein R₃ is each independently selected from the group consisting of H and an alkyl of 1 to 4 carbon atoms, and
i) COR₁ wherein R₁ is selected from the group consisting of H, (CH₂)n CH₃ wherein n=0-6, (CH₂)n COOR₂ wherein n=1-4 and R₂ is each selected from the group consisting of H, an alkali metal salt, an alkaline earth metal salt, NH₄+ and N+(R₃)₄, and (CH₂)n N+(R₃)₄, wherein n=1-4 and R₃ is each independently selected from the group consisting of H and an alkyl of 1 to 4 carbon atoms.

3. The method of claim 1, wherein R is selected from the group consisting of H and acetyl.

4. The method of claim 1, wherein R is acetyl.

5. The method of claim 1, wherein the compound is delivered in a daily dose of between about 1 mg to about 4000 mg.

6. The method of claim 1, wherein the compound is administered along with a standard chemotherapy regimen.

7. The method of claim 6, wherein the standard chemotherapy regimen is selected from the group consisting of TNP-470, aspirin, salicylate, angiostatin, endostatin, Bay-129566 and thalidomide.

8. A method of prevention and/or treatment of obesity or an overweight condition comprising:
delivering an effective amount of hypoestes rosea dried leaf powder.

9. The method of claim 8, wherein the hypoestes dried leaf powder is delivered in a daily dose of between about 1 mg to about 4000 mg.

10. The method of claim 8, wherein the hypoestes dried leaf powder is administered along with a standard chemotherapy regimen.

11. The method of claim 10, wherein the standard chemotherapy regimen is selected from the group consisting of TNP-470, aspirin, salicylate, angiostatin, endostatin, Bay-129566 and thalidomide.
